(19) United States
(12) Patent Application Publication
(10) Pub. No.: US 2025/0200836 A1
(43) Pub. Date: Jun. 19, 2025

(54) Causal Device and Causal Method Thereof

(71) Applicant: Wistron Corporation, New Taipei City (TW)

(72) Inventor: Chih-Ming Chen, New Taipei City (TW)

(73) Assignee: Wistron Corporation, New Taipei City (TW)

(21) Appl. No.: 18/616,105

(22) Filed: Mar. 25, 2024

(30) Foreign Application Priority Data
Dec. 18, 2023 (TW) 112149260

(51) Int. Cl. G06T 11/00

(52) U.S. Cl. CPC G06T 11/005; G06T 2210/41; G06T 2211/441

(57) ABSTRACT

A causal device, which includes a causal module and a causal feature learning module coupled to the causal module, and a causal method thereof is disclosed to ensure accurate fusion of hybrid imaging or improve priority triage of imaging tests. The causal module is configured to identify or utilize causal relationship(s) between a plurality of variables; the causal feature learning module is configured to extract at least one first causal feature of one of the plurality of variables.

### BACKGROUND OF THE INVENTION

### 1. Field of the Invention

The present invention relates to a causal device and causal method thereof, and more particularly, to a causal device and causal method thereof to ensure accurate fusion of hybrid imaging or improve priority triage of imaging tests.

### 2. Description of the Prior Art

Hybrid imaging, which refers to the combination of two or more imaging modalities (e.g., PET/MRI or PET/CT) in a single imaging session to obtain complementary information about the anatomy and function of the imaged tissue or organ, can provide more accurate and comprehensive information than either modality alone. One of the main challenges in PET/MRI imaging is accurate attenuation correction, as MRI images do not provide direct information about photon attenuation. The attenuation map for PET/MRI is typically generated using a combination of methods; however, these methods can be susceptible to errors, particularly in regions with high tissue heterogeneity or metal implants. Another challenge in PET/MRI imaging is the correction of motion and registration errors between the PET and MRI images: PET and MRI images are often acquired separately and then registered to each other, which can introduce errors and misalignment due to differences in the imaging geometry and physiological state. In addition, PET/MRI images can be affected by various artifacts (e.g., radiofrequency interference, susceptibility, and chemical shift artifacts), which is also a challenge in PET/MRI imaging. While PET/MRI imaging offers many advantages over PET/CT (Positron Emission Tomography/Computed Tomography) imaging, such as improved soft-tissue contrast and reduced radiation exposure, there are several challenges that need to be addressed to ensure the accuracy and reliability of the imaging data.

Priority triage involves the process of prioritizing patients based on the severity of their condition and the urgency of their need for medical attention. For example, priority triage in the context of medical imaging is used to determine the urgency of a patient's need for further diagnostic imaging (e.g., a CT scan) based on the findings of previous imaging studies (e.g., X-rays). However, existing priority triage methods for medical imaging are not always accurate in predicting which patients require immediate attention, leading to delays in treatment for some patients and unnecessary interventions for others. Moreover, many existing priority triage methods are based on fixed rules or algorithms that cannot be easily adapted to different patient populations or clinical settings, resulting in suboptimal performance in certain situations. Existing priority triage methods often rely on a limited set of clinical features or imaging modalities, which may not capture the full complexity of a patient's condition. There may be the potential for biases to be introduced and ethical concerns, particularly if existing priority triage methods are used to determine access to limited resources (e.g., imaging equipment or critical care beds) but the algorithms are not designed and validated appropriately. Many existing priority triage methods require input from trained radiologists or other healthcare professionals to interpret imaging results and make decisions about patient prioritization, which can be time-consuming and resource-intensive.

### SUMMARY OF THE INVENTION

It is therefore a primary objective of the present invention to provide a causal device and causal method thereof, to improve over disadvantages of the prior art.

The present invention discloses a causal device, comprising a causal device, comprising a causal module, configured to identify or utilize causal relationships between a plurality of variables; and a causal feature learning module, coupled to the causal module, configured to extract at least one first causal feature of one of the plurality of variables.

The present invention discloses a causal method, for a causal device, comprising identifying or utilizing causal relationships between a plurality of variables; and extracting at least one first causal feature of one of the plurality of variables.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of an image reconstruction device according to an embodiment of the present invention.

FIG. 2 is a schematic diagram of an image reconstruction method according to an embodiment of the present invention.

FIG. 3 is a schematic diagram of a causal graph corresponding to SCM according to an embodiment of the present invention.

FIG. 4 is a schematic diagram of a causal graph corresponding to CTSCM according to an embodiment of the present invention.

FIG. 5 is a schematic diagram of a CFL module according to an embodiment of the present invention.

FIG. 6 is a schematic diagram of a reconstruction module according to an embodiment of the present invention.

FIG. 7 is a schematic diagram of a priority triage device according to an embodiment of the present invention.

FIG. 8 is a schematic diagram of a priority triage method according to an embodiment of the present invention.

FIG. 9 is a schematic diagram of a CTSCM according to an embodiment of the present invention.

FIG. 10 is a schematic diagram of a causal device according to an embodiment of the present invention.

### DETAILED DESCRIPTION

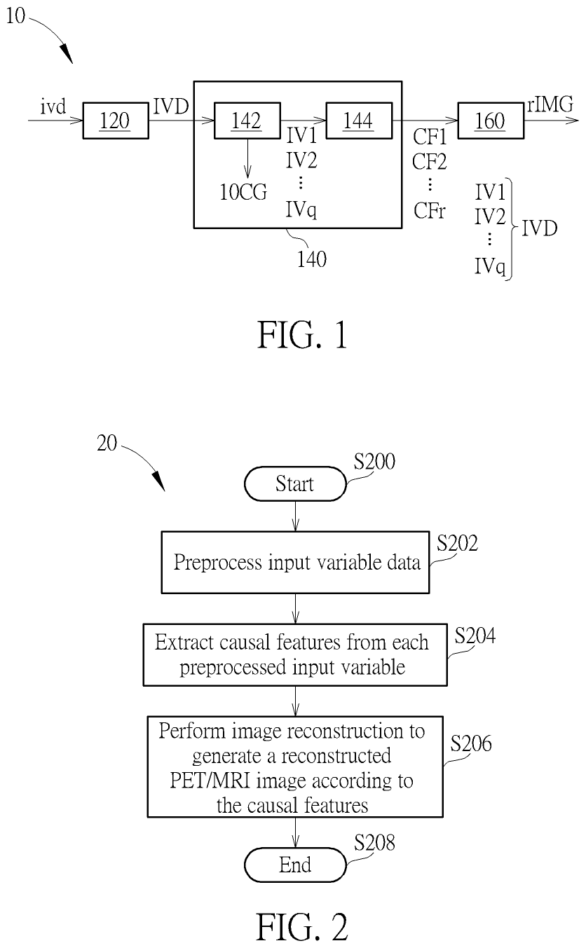

FIG. 1 is a schematic diagram of an image reconstruction device 10 according to an embodiment of the present invention. The image reconstruction device 10 may comprise a preprocessing module 120, an extraction module 140 and a reconstruction module 160. The extraction module 140 may comprise a causal inference module 142 and a causal feature learning (CFL) module 144.

The preprocessing module 120 may perform necessary preprocessing on input variable data ivd (referred to as first input variable data) to generate input variable data IVD (referred to as second input variable data). The (preprocessed) input variable data IVD, which is converted from the input variable data ivd, may comprise input variables IV1-IVq, which may have different types and dimensions. In one embodiment, the input variable data ivd or IVD may comprise, for example, PET (Positron Emission Tomography) data (e.g., PET sinogram(s) or PET image(s)), MRI (Magnetic Resonance Imaging) data (e.g., MRI sequence(s) or MRI image(s)), patient demographics, imaging protocol(s), or scanner characteristic(s). PET sinograms are raw data obtained from PET scans and are used to create/reconstruct PET images. MRI sequences refer to a set of MRI images acquired at different times during scanning.

The causal inference module 142 may be used to identify the causal relationship(s)/causality 10CG between the preprocessed input variable(s) IV1-IVq and an outcome variable OV. In one embodiment, the outcome variable OV may be or be related to a reconstructed PET/MRI image. The CFL module 144 may be used to extract causal features CF1-CFr from the preprocessed input variables IV1-Ivq. The causal feature CF1 . . . or CFr may, for example, correspond to or comprise certain information or a certain part of an image, but is not limited thereto. The reconstruction module 160 may be used to generate a reconstructed PET/MRI image rIMG based on the causal features CF1-CFr.

In a word, a reconstructed PET/MRI image (e.g., rIMG) refers to a PET/MRI image generated through an image reconstruction algorithm using input variable data such as PET sinogram(s) and MRI sequence(s). PET sinogram(s) and MRI sequence(s) may be registered or corrected; for example, MRI image(s) may provide anatomical information for PET image (reconstruction) through attenuation correction. In this case, at least the PET sinogram(s) and the MRI sequence(s) have causal relationships with and are causally related to the reconstructed PET/MRI image. The causal inference module 142 may learn/determine the causal relationship(s) 10CG. The CFL module 144 may extract the causal features CF1-CFr corresponding to the causal relationship(s) 10CG. The reconstruction module 160 generates the reconstructed PET/MRI image rIMG based on the causal features CF1-CFr. In other words, the causal features CF1-CFr imply features that affect/cause the outcome variable OV. Therefore, compared with the existing hybrid imaging PET/MRI images, the reconstructed PET/MRI image rIMG is more accurate.

FIG. 2 is a schematic diagram of an image reconstruction method 20 according to an embodiment of the present invention. The image reconstruction method 20 is suitable for the image reconstruction device 10 and may comprise the following steps:

Step S200: Start.

Step S202: Preprocess input variable data (e.g., ivd), which comprises input variables.

Step S204: Extract causal features (e.g., CF1, . . . , or CFr) from each preprocessed input variable (e.g., IV1 to IVq).

Step S206: Perform image reconstruction according to the causal features to generate a reconstructed PET/MRI image (e.g., rIMG). (For example, use GAN to reconstruct a PET/MRI image).

Step S208: End.

The image reconstruction method 20 is further described below. In step S202, the image reconstruction device 10 may receive the input variable data (e.g., ivd), which may be loaded into memory of the image reconstruction device 10.

In one embodiment, in step S202, the preprocessing module 120 may perform any necessary preprocessing such as attenuation correction, motion correction, registration, standardization, or normalization on the input variable data (e.g., ivd) or input variables.

In one embodiment, attenuation correction of PET sinogram(s) may use a transmission-based attenuation correction (TAC) method, which may be more accurate or quantitative or facilitate no dependence on external factors such as patient size or body habitus. The additional radiation exposure from a transmission scan is relatively low compared to the radiation exposure from the PET scan itself. Motion correction of PET sinogram(s) may use a motion-compensation reconstruction (MCR) method, which may achieve accurate motion correction even for large motion and may be implemented with or without access to motion tracking data. Normalization or standardization of PET sinogram(s) may correct for variations in scanner sensitivity and other factors that can affect the image quality, and may use standard uptake value (SUV) method.

In one embodiment, motion correction of MRI sequence(s) may use a non-rigid registration method to handle non-linear deformations.

In one embodiment, registration of PET sinogram(s) or MRI sequence(s) may use a gradient correlation (GC) method, which may be robust to intensity differences or noise. Registration between PET sinogram(s) and MRI sequence(s) in PET/MRI image(s) may refer to the process of aligning the PET sinogram(s) and the MRI sequence(s) to the same coordinate space to facilitate their joint analysis. PET sinogram(s) and MRI sequence(s) are acquired in different imaging modalities, and spatial resolution or image quality of PET sinogram(s) and MRI sequence(s) may be different. Thus, registration is required to compensate for the differences and enable accurate fusion of the two imaging modalities for better diagnosis and treatment planning.

In one embodiment, normalization or standardization of patient demographics may remove any confounding effects of demographic variables on the imaging data, and may use a robust scaling method, which is robust to outliers or preserves the distribution of data. Patient demographics may comprise gender or age.

In one embodiment, normalization or standardization of imaging protocol may use a use-of-phantom-studies method, which may be used to validate and optimize imaging protocols, or may allow for ongoing monitoring of imaging quality.

In one embodiment, preprocessing for scanner characteristics may be performed by the scanner manufacturer.

In one embodiment, in step S202, the image reconstruction device 10 (e.g., the preprocessing module 120) may further divide/split the preprocessed input variable data into a training set and a testing set.

In one embodiment, at least part of the image reconstruction method 20 may be compiled into a pseudocode. For example, corresponding to step S202, the pseudocode may comprise:

```
# Step 1: Preprocessing
  # PET data preprocessing
  pet_data = load_pet_data(pet_file)
  pet_data = apply_attenuation_correction(pet_data) # optional
  pet_data = apply_motion_correction(pet_data) # optional
  pet_data = apply_registration(pet_data) # optional
  pet_data = apply_normalization_standardization(pet_data) # optional
  # MRI data preprocessing
  mri_data = load_mri_data(mri_file)
  mri_data = apply_motion_correction(mri_data) # optional
  mri_data = apply_registration(mri_data, reference_image) # optional
  # Patient demographics data preprocessing
  pat_dem_data = apply_normalization_standardization (pat_dem_data) # optional
  # Imaging protocol data preprocessing
  ima_pro_data = apply_normalization_standardization (ima_pro_data) # optional
```

In one embodiment, in step S202, since each input variable data is collected at a specific time instant, the input variable data may be discrete, but it may be converted into continuous-time input variable data using (linear or nonlinear) interpolation method(s).

In step S204, the causal inference module 142 may apply a causal inference algorithm to identify/determine the (underlying) causal relationship(s) (e.g., 10CG) between the preprocessed input variable(s) (e.g., IV1 . . . or IVq) and the outcome variable (e.g., OV).

In one embodiment, the causal inference algorithm may comprise, for example, a continuous time structured equation modeling (CTSEM) framework. In one embodiment, the causal inference algorithm may be applied to a continuous time structural causal model (CTSCM), which is a machine learning framework that can learn the causal relationship(s) between preprocessed input variable(s) and the outcome variable. In one embodiment, the CTSEM framework may be part of the CTSEM. In one embodiment, CTSCM may be processed using software such as Python, Gephi, DAGitty, or other software; CTSEM may be analyzed using software such as LISREL, Knitr, OpenMx, Onyx, Stata, or other software.

In one embodiment, the causal inference module 142 in step S204 may define/generate a causal graph, which defines/presents/describes the causal relationship(s) between the preprocessed input variable(s) and the outcome variable. In one embodiment, the causal graph may be drawn using domain knowledge or previous research.

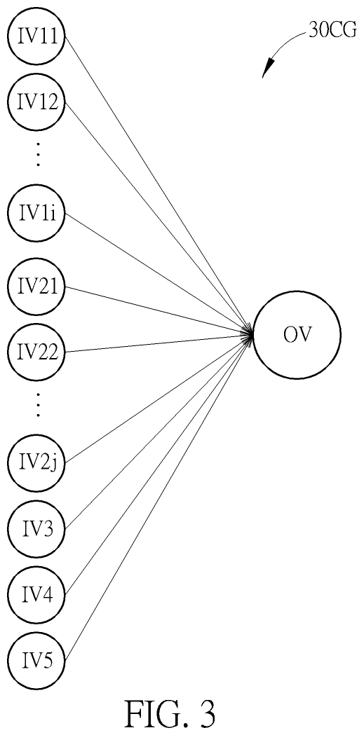

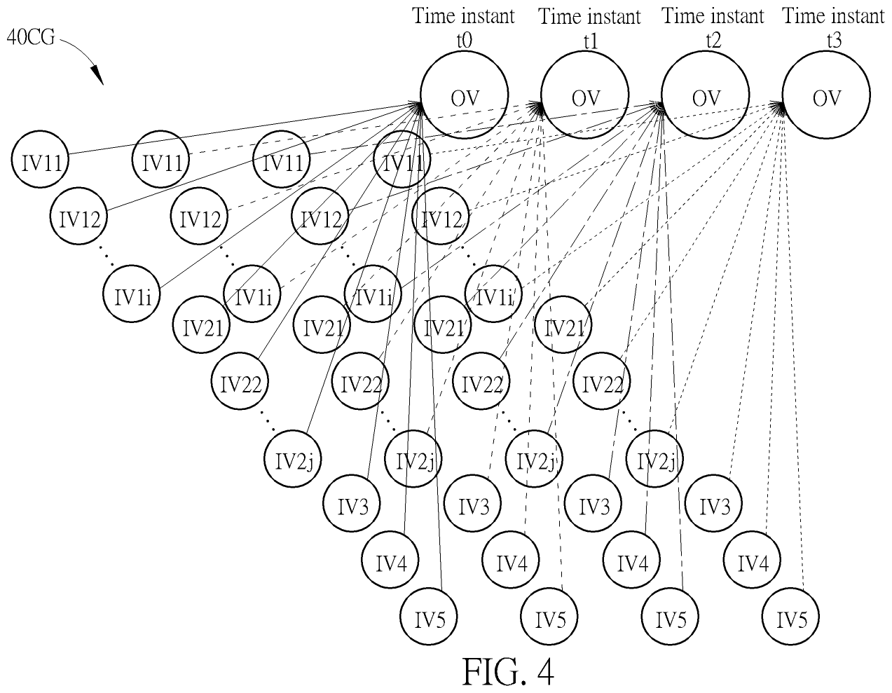

For example, FIG. 3 is a schematic diagram of a causal graph 30CG corresponding to SCM according to an embodiment of the present invention; FIG. 4 is a schematic diagram of a causal graph 40CG corresponding to CTSCM according to an embodiment of the present invention. In FIGS. 3 and 4, the preprocessed input variables IV1-Ivq may be implemented using preprocessed input variables IV11-IV5. In one embodiment, the preprocessed input variables IV11-IV1i may be/represent/correspond to PET sinograms at different angles, respectively. The preprocessed input variables IV21-IV2j may be/represent/correspond to T1-weighted or T2-weighted MRI sequences, respectively. The preprocessed input variables IV3-IV5 may be/represent/correspond to patient demographics, an imaging protocol, and scanner characteristic(s), respectively. In other words, the causal graph 30CG or 40CG may provide insight into the causal relationship(s) (e.g., 10CG) between the input variable(s) and the outcome variable OV.

In FIGS. 3 and 4, the causal graph 30CG may describe the causal relationships between the preprocessed input variables IV11-IV5 and the outcome variable OV, and the causal graph 40CG may describe the causal relationships between the preprocessed input variables IV11-IV5 and the outcome variable OV at different time instants t0-t3. FIGS. 3 and 4 show that SCM or its causal graph 30CG only corresponds to one specific moment of CTSCM or its causal graph 40CG, while CTSCM may comprise/correspond to numerous moments. In other words, CTSCM may be regarded as comprising multiple SCMs, and CTSCM may capture the causal relationships between the preprocessed input variables IV11-IV5 and the outcome variable OV over time. Similarly, SEM frame only analyzes one specific moment of the CTSEM frame.

Although the causal graph 40CG only describes the causal relationships between the preprocessed input variables IV11-IV5 and the outcome variable OV at a time instant (e.g., t0), in one embodiment, the outcome variable OV at the time instant t1 may be influenced by the input variables at the time instant t0. The exact nature and strength of causal relationship(s) depend(s) on the specific imaging protocol, patient characteristics, or other factors, and may vary from case to case.

In one embodiment, in step S204, once a CTSCM has been selected/determined, the CTSCM may be trained using the preprocessed input variables (and the outcome variable). During training, the CTSCM may learn the causal relationships between the preprocessed input variable(s) and the reconstructed PET/MRI image, which are used for subsequent causal feature extraction (step S204) (to extract causal features from the preprocessed input variables) and for subsequent image reconstruction (step S206), allowing for more accurate and robust image reconstruction.

In one embodiment, in step S204, training of CTSCM involves finding model parameter value(s) that best fit the preprocessed input variables (and the outcome variable). The algorithm used to train CTSCM may comprise maximum likelihood estimation (MLE), Bayesian inference, expectation-maximization (EM), or other algorithms. MLE is a statistical method used to find model parameter value(s), which maximize the probability of observed data (e.g., the input variable(s) or the outcome variable). MLE is relatively easy to implement and is often effective at finding model parameter value(s) that produce good predictions. Bayesian inference is a statistical method used to train CTSCM by iteratively updating model parameter value(s) as new preprocessed input variables (and outcome variable) are collected. EM is an iterative algorithm used to find model parameter value(s), which maximize the likelihood of observed data (e.g., the input variable(s) or the outcome variable). EM is effective at handling the observed data which is incomplete or comprises missing values. The choice of which algorithm to use to train a CTSCM may depend on a variety of factors, such as the specific characteristics of the model, the size/quality of the available input variables (or outcome variable), and the computational resources that are available.

For example, the image reconstruction method 20 may also involve training a CTSCM using MLE, and may further comprise the following steps:

Step S500: Start.

Step S502: Initialize model parameter value(s) of a CTSCM. In one embodiment, it may be done by randomly generating the model parameter value(s) or by using expert knowledge to set the initial value(s). Next, proceed to step S504.

Step S504: Use the CTSCM to predict the predicted value(s) of variable(s) (e.g., the outcome variable OV, the input variable IVq, the causal relationship(s) 10CG, probabilities, or conditional probabilities). Next, proceed to step S506.

Step S506: Compare the predicted value(s) of the variable(s) with the actual/real value(s) of the variable(s) (e.g., a PET/MRI image serving as the ground truth, the actual/real input variable IVq, the causal relationship, probabilities, or conditional probabilities). Next, proceed to step S508.

Step S508: Update the model parameter value(s) so that the predicted value(s) of the CTSCM is/are closer to the actual/real value(s).

Step S510: Determine whether the model parameter value(s) change(s) significantly. If there is a significant change, proceed to step S504; if not, proceed to step S512.

Step S512: End.

For example, in step S204, the image reconstruction device 10 obtains a set of PET sinogram(s) and MRI sequence(s), as well as patient demographics, imaging protocol(s), and scanner characteristic(s) related to the PET sinogram(s) and MRI sequence(s). The image reconstruction device 10 may use the preprocessed input variables (e.g., IV1 . . . or IVq) to train CTSCM to understand the causal relationship(s) between the preprocessed input variable(s) and the reconstructed PET/MRI image. Alternatively, for example, in step S204, the image reconstruction apparatus 10 may use CTSCM to learn the causal relationship(s) between the PET sinogram(s) and the MRI sequence(s). The PET sinogram(s) may be used as the preprocessed input variables, and MRI sequence(s) may be used as the outcome variable. By training CTSCM, CTSCM may learn how the PET sinogram(s) affect the MRI sequence(s), and thus understand the correlation between the PET sinogram(s) and the MRI sequence(s). The training of CTSCM involves training using the preprocessed input variables and the outcome variable to understand the causal relationship(s) between the preprocessed input variable(s) and the outcome variable.

In one embodiment, CTSCM may comprise model parameter value(s) that is/are time-dependent, so the training of CTSCM may require more data of input variable(s) to determine the function of the model parameter value(s) with respect to time. In one embodiment, depending on whether the time-dependent model parameter value(s) of CTSCM is/are linear or nonlinear, linear regression model(s) (e.g., θ(t)=α+βt, where θ(t) represents a model parameter value at a time instant t, and a represents its initial model parameter value) or nonlinear regression model(s) (e.g., θ(t)=f(t), where f(t) represents a nonlinear function) may be used to estimate the model parameter value(s) as function(s) of time.

In step S204, the CFL module 144 may use a CFL algorithm to extract causal feature(s) from each preprocessed input variable. The causal feature(s) may describe the causal relationship(s) between the preprocessed input variable(s) and the outcome variable. In general, a causal feature can be more robust to noise than an ordinary feature, especially if the noise affects the statistical regularities in the data but not the underlying causal relationship(s) between the input variable(s) and the outcome variable. Causal feature(s) may be designed to capture the underlying cause(s) that give rise to the observed data (e.g., the input variable(s) or the outcome variable), and not just the statistical regularities in the data itself, which can make it/them more robust to noise in some cases.

In one embodiment, the CFL algorithm may be an unsupervised CFL algorithm or a CFL neural network. In one embodiment, the CFL algorithm may be a CFL neural network in the form of CTSCM. In other words, CTSCM may be a machine learning framework that can extract causal feature(s) from the preprocessed input variable(s) using an unsupervised CFL algorithm. Extracting causal feature(s) from input variable(s) using CTSCM refers to identifying the underlying causal relationship(s) between the preprocessed input variable(s) and the outcome variable. The causal feature(s) obtained from CTSCM provide(s) insights into the mechanisms that govern the causal relationship(s) between the preprocessed input variable(s) and outcome variable, and improve(s) the accuracy and robustness of image reconstruction.

In one embodiment, the CFL algorithm may learn disentangled and causally relevant features by minimizing the mutual information between the latent variables at different time steps, while maximizing the mutual information between the latent variables and the observed variables. In one embodiment, the causal feature(s) extracted by the CFL algorithm may be used to cluster input variables and improve the accuracy of reconstructed PET/MRI image.

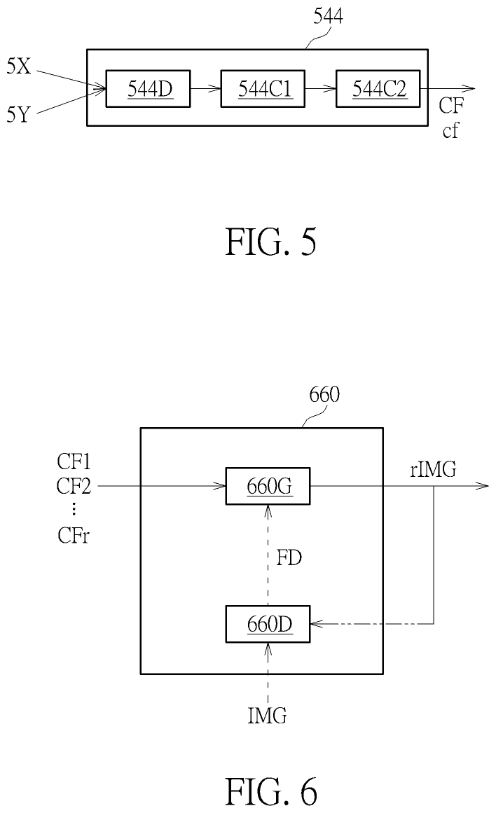

For example, FIG. 5 is a schematic diagram of a CFL module 544 according to an embodiment of the present invention. The CFL module 144 may be implemented using the CFL module 544. The CFL module 544, which may be used to execute the CFL algorithm, and may comprise at least one density estimation block 544D and at least one clustering block 544C.

In one embodiment, the density estimation block 544D may be used to receive data 5X, 5Y, which comprise macrovariables, and may estimate probability density function(s) of the data 5X or 5Y (e.g., the outcome variable OV, the input variable(s) IV1 . . . . IVq, IV11 . . . or IV5). In one embodiment, the density estimation block 544D may calculate/estimate, for example, conditional probability P(5Y|5X), which is a measure of the probability of the data 5Y occurring, given that the data 5X is already known to have occurred.

In one embodiment, clustering block 544C is used to partition data into different clusters. In one embodiment, the clustering block 544C may be divided at least into clustering blocks 544C1 and 544C2. The clustering block 544C1 may divide the data 5X into different clusters according to the conditional probability P(5Y|5X), so that the data 5X that have similar prediction(s) for the data 5Y are grouped into the same cluster. The clustering block 544C2 may divide the data 5Y into different clusters according to the conditional probability P(5Y|5X), so that the data 5Y that have similar response(s) to any intervention are grouped into the same cluster. In one embodiment, the data of the same cluster may correspond to one or more causal features, so that the CFL module 544 may use the CFL algorithm to extract at least one causal feature (e.g., CF or cf) from each preprocessed input variable.

In the CFL algorithm, density estimation and clustering are the two main steps used to generate macrovariables. The macrovariables may be interpreted as meaningful scientific quantities and used for causal interpretation. In the CFL algorithm, density estimation is the first step in generating macrovariables, and clustering is the second step. Therefore, the CFL module 544 encapsulates a series of blocks covering the major categories of the CFL algorithm and coordinates/orchestrates a data transformation pipeline.

In one embodiment, the CFL algorithm used by the CFL module 544 may comprise, for example, Table 1:

TABLE 1

```
input: D = {(x₁, y₁), ... ,(x_{N}, y_{N})}
  CDEModel - a conditional density estimation method
  CClusteringModel - a clustering method for cause space
  EClusteringModel - a clustering method for effect space
output: xlbls, ylbls - the macrovariable classes of each x, y.
  Estimate f ← CDEModel(D; loss_fxn = Σᵢ(f(xᵢ) − yᵢ)²);
  Let xlbls + CClusteringModel(f(x₁), ... ,f(x_{N}));
  Let Y_{w} ← {y | xlbls = w and (x, y) ∈ D};
  Let g(y) ← [kNN(y,Y₀), ... , kNN(y,Y_{w})]
  Let ylbls ← EClusteringModel(g(y₁), ... , g(y_{N}));
```

In one embodiment, the unsupervised CFL algorithm used by the CFL module 544 is suitable for Discrete Time Structural Causal Models (DTSCM). In one embodiment, the unsupervised CFL algorithm may be adapted for use with CTSCM by incorporating time-dependent parameter(s) or modifying the optimization objective to account for continuous time dimension. For example, for continuous time input variable(s), the following methods one to four may be used according to the nature of the input variable(s) and the complexity of the CFL algorithm to consider continuous time dimension, thereby improving accuracy and flexibility.

For example, in method one, the CFL algorithm may be modified to comprise parameter(s) that change(s) over time so as to incorporate time-dependent parameter(s) into the CFL algorithm (for CTSCM). In one embodiment, this may be done by using a dynamic model of the system (e.g., a differential equation model). The time-dependent parameter(s) may then be estimated using the same optimization procedure as the other parameters of the CFL algorithm. In one embodiment, the CFL algorithm may treat parameter(s) as a function of time. For example, a linear function (e.g., θ(t)=α+βt, where θ(t) represents the value of a parameter at a time instant t, and a represents the initial value of the parameter) or a nonlinear function (e.g., θ(t)=f(t), where f(t) represents a nonlinear function) may be used to represent the change of parameter(s) over time to incorporate time-dependent parameter(s) into the CFL algorithm. In one embodiment, a parameter model that changes over time may be used to represent the influence of PET sinogram(s) and MRI sequence(s) on a reconstructed PET/MRI image. For example, the impact of PET sinogram(s) on a reconstructed PET/MRI image may be regarded as a function of time.

For example, in method two, an error model that changes over time may be used. In one embodiment, the CFL algorithm may treat an error as a function of time. For example, a noise model may be used to represent the change of the error over time to incorporate time-dependent parameter(s) into the CFL algorithm. For example, noise in PET sinogram(s) or MRI sequence(s) may be treated as a function of time. For example, the noise model may satisfy y (t)=0 (t)+c (t), where y(t) represents the value of an input variable at a time instant t, and ¿ (t) represents the error at the time instant t.

For example, in method three, a time-dependent loss function may be used to modify the optimization objective for the CFL algorithm in the continuous time setting, thereby accounting for continuous time dimension. The loss function may be related to the optimization objective. The loss function may be a specific measure of how well the CFL algorithm is performing on a particular task; the optimization objective may be the overall goal of the CFL algorithm. The optimization objective is to learn a model that accurately captures the underlying causal relationships between the preprocessed input variables IV1-IVq and the outcome variable in a continuous time setting. The time-dependent loss function may be designed to encourage the CFL algorithm to learn causal feature(s) that is/are disentangled and causally relevant, even in the presence of time-varying signals, so that the loss function indirectly affects the mutual information between latent variables or between latent variable(s) and observed variable(s) (e.g., input variable(s)). For example, the loss function may be designed to minimize the mutual information between the latent variables at different time steps, while maximizing the mutual information between the latent variables and the observed variables, where the latent variables may be part of the input or output of the CTSCM, rather than the CFL algorithm or its blocks. In one embodiment, the time-dependent loss function may comprise a combination of the standard loss function used in the CFL algorithm (e.g., mean squared error) and a time-dependent regularization term. The exact form of the time-dependent loss function may depend on the specific application, the properties of the input variable data being analyzed, and the desired properties of the learned causal feature(s).

For example, in method four, a time-dependent regularization term may be used to modify the optimization objective for the CFL algorithm in the continuous time setting, thereby accounting for continuous time dimension. The regularization term may be a specific penalty term that may be added to the optimization objective and may be used to penalize the CFL algorithm for learning causal feature(s) that is/are not causally relevant or that is/are not invariant to time-shifts. In other words, the regularization term may be used to discourage the CFL algorithm from learning causal feature(s) unimportant for predicting the outcome variable, causal feature(s) that change(s) over time in a way that is not related to the outcome variable, or causal feature(s) inconsistent across different time instants. For example, the regularization term may be designed to penalize the model for learning causal feature(s) that are correlated with the time derivative of the observed variable(s). In one embodiment, the loss function may comprise a time-dependent regularization term. The exact form of the time-dependent regularization term may depend on the specific application, the properties of the input variable data being analyzed, the CFL algorithm, and the causal feature(s) being learned.

For example, the image reconstruction method 20 may also involve modifying the CFL algorithm used by the CFL module 544 for CTSCM, and may further comprise the following steps:

Step S600: Start.

Step S602: Use a dynamic model (e.g., a differential equation model) to perform modeling. Next, proceed to step S604.

Step S604: Define a time-dependent loss function that encourages the CFL algorithm to learn disentangled and causally relevant feature(s). Next, proceed to step S606.

Step S606: Define a time-dependent regularization term that penalizes the CFL algorithm for learning feature(s) that are not causally relevant or that are not invariant to time-shifts. Next, proceed to step S608.

Step S608: Train the CFL algorithm using the modified optimization objective.

Step S610: End.

In one embodiment, the differential equation model may involve stochastic differential equations and may satisfy ηₕ(t)=e^{A(t-t}^{₀}⁾ ηₕ(t₀)+A⁻¹[e^{A(t-t}^{₀}⁾−I]ξₕ+A⁻¹[e^{A(t-t}^{₀}⁾−I]Bzₕ+M Σᵤx_{h,u}δ(t−u)+∫ₜ_{₀}^{t}e^{A(t-s)}GdWₕ(s) (Equation 1), or dηₕ(t)=(Aηₕ(t)+ξₕ+Bzₕ+MΣᵤx_{h,u}δ(t−u))dt+GdWₕ(t) (Equation 2). The vector ηₕ(t) may be a function of time and may be used to implement model parameter value(s), parameter(s), error(s), a loss function, or a regularization term. The matrix A may use auto effects on the diagonal and cross effects on the off-diagonal to qualitatively characterize/capture the temporal relationships of the vector nₕ(t). The matrix I is the identity matrix. The random vector ξₕ may determine the long-term trend/level of the vector ηₕ(t) and may follow/satisfy a distribution ξₕ˜N(κ, ϕ_{ξ}), where the vector κ may represent continuous time intercept(s), and the matrix ϕ_{ξ} may represent a covariance. The matrix B may represent the effect of a (fixed) time-independent predictor vector zₕ on the vector ηₕ(t), and the number of rows of the matrix B may differ from the number of columns of the matrix B. The time-dependent predictor vector x_{h,u} may be observed at a time instant u and may be treated as impacting the vector ηₕ(t) only at the time instant u, and the effect of impulses, each of which is formed/described by x_{h,u}δ(t−u), on the vector ηₕ(t) may be represented by the matrix M. The vectors Wₕ(s) may be independent random walks in continuous time (e.g., Wiener processes), and dWₕ(s) may be a stochastic error term. The lower triangular matrix G may represent the effect on changes of the vector ηₕ(t). The matrix Q satisfying Q=GG^{T} may represent a variance-covariance matrix of the diffusion process in continuous time. In one embodiment, CTSCM may also satisfy Equation 1 or Equation 2.

In one embodiment, in step S204, the CFL algorithm may convert discrete variable(s) into continuous variable(s), so the CFL algorithm may be used to preprocess the input variable(s) of CTSCM, and the continuous input variable(s) may serve as the input of CTSCM, enabling CTSCM to learn the causal relationship(s) between preprocessed input variable(s) and a reconstructed PET/MRI image. The CFL algorithm may first calculate the empirical distribution of discrete variables, then use the empirical distribution to generate continuous variables with the same distribution as the discrete variables, and repeat the above process for each discrete variable of the input variable data. Since CTSCM relies on accurate and consistent input to understand the causal relationship(s) between preprocessed input variable(s) and a reconstructed PET/MRI image, converting discrete variable(s) into continuous variable(s) ensures that the input variables meet the requirements of CTSCM.

In step S204, the image reconstruction device 10 (e.g., the extraction module 140) may combine (e.g., concatenate) causal features of all input variables to establish a unified set of causal features for input variable data IVD (e.g., PET image(s) or MRI image(s)).

In one embodiment, corresponding to step S204 of the image reconstruction method 20, a pseudocode may comprise:

```
# Step 2: Extract causal features
  # PET feature extraction for signograms
  pet_features = extract_causal_features(pet_data)
  # MRI feature extraction for sequences
  mri_features = extract_causal_features(mri_data)
  # Patient demographics feature extraction
  Pat_dem_features = extract_causal_features(pat_dem_data)
  # Imaging protocol feature extraction
  ima_pro_features = extract_causal_features(ima_pro_data)
  # Scanner characteristics feature extraction
  sca_cha_features = extract_causal_features(sca_cha_data)
```

In step S206, the reconstruction module 160 may use input variable data (e.g., PET image(s) or MRI image(s)), which has been preprocessed and causal-feature extracted, from the training set to train a Generative Adversarial Network (GAN) model. The PET/MRI image(s) of ground truth or the existing hybrid imaging PET/MRI image(s) may be used as label(s). The reconstruction module 160 may utilize a GAN model as an image reconstruction algorithm and generate high-quality reconstructed PET/MRI image (e.g., rIMG) with fine details based on causal features (e.g., CF1 . . . or CFr) extracted from the preprocessed input variables (e.g., IV1 . . . or IVq), making them potentially useful for applications where visual accuracy is important. Using a GAN model as an image reconstruction algorithm may facilitate learning for generating a reconstructed PET/MRI image from highly complex and diverse data distributions, making it useful in situations where traditional reconstruction methods may struggle. Using a GAN model as an image reconstruction algorithm may potentially reduce the amount of data needed for reconstruction, since it can fill in missing data or interpolate between existing data points.

For example, FIG. 6 is a schematic diagram of a reconstruction module 660 according to an embodiment of the present invention. The reconstruction module 160 may be implemented using the reconstruction module 660, which may comprise a generator network 660G and a discriminator network 660D. The generator network 660G or the discriminator network 660D may be a neural network (NN).

In one embodiment, during training, the generator network 660G and the discriminator network 660D may be trained together (in an adversarial process). In one embodiment, the generator network 660G may receive/utilize the input variable data IVD, which has been preprocessed and causal-feature extracted, (or a random noise vector, the causal features CF1 . . . or CFr extracted by the CFL module 144) in step S206, and attempt to generate a reconstructed PET/MRI image rIMG1 used to deceive the discriminator network 660D. The random noise vector is used to introduce stochasticity into the generator network 660G and help it generate diverse and realistic reconstructed PET/MRI image(s) (e.g., rIMG1). The generator network 660G takes its inputs and generates reconstructed PET/MRI image(s) (e.g., rIMG1) that may or may be intended to be similar to real PET/MRI image(s) (e.g., IMG) of ground truth in terms of causal relationships.

In one embodiment, during training, the discriminator network 660D is used to receive the real PET/MRI image IMG (or the PET/MRI image IMG of the existing hybrid imaging) and the reconstructed PET/MRI image rIMG1 generated by the generator network 660G. The discriminator network 660D may be used to learn/distinguish between the real PET/MRI image and the reconstructed PET/MRI image by comparing the features (or visual appearance) of the real PET/MRI image IMG and the reconstructed PET/MRI image rIMG1, and assigning a probability score to the reconstructed PET/MRI image rIMG1 to indicate how likely the reconstructed PET/MRI image rIMG1 is to be real. The discriminator network 660D may not require direct knowledge of causal feature(s). The discriminator network 660D may evaluate the reconstructed PET/MRI image rIMG1 and provide feedback FD to the generator network 660G to improve performance. The generator network 660G learns to produce the reconstructed PET/MRI image rIMG1 that are increasingly similar/closer to the real PET/MRI image IMG over time, and the discriminator network 660D may become more accurate in distinguishing between the real PET/MRI image IMG and the reconstructed PET/MRI image over time.

In step S206, the reconstruction module 160 may also use the trained GAN model to synthesize image(s) according to the causal feature(s) of the testing set so as to generate a reconstructed PET/MRI image rIMG2. In one embodiment, during testing, the generator network 660G may takes as input the input variable data IVD, which has been preprocessed and causal-feature extracted, (or the causal features CF1 . . . or CFr extracted from the preprocessed input variable(s) by the CFL module 144) in step S206, and use it to generate the reconstructed PET/MRI image rIMG2. The reconstructed PET/MRI image rIMG2 may be similar to the real PET/MRI image IMG in terms of causality, which helps to improve the accuracy and robustness of image reconstruction.

In other words, the causal feature(s) extracted by CTSCM (e.g., CF1 . . . or CFr) may be input to the GAN model, and the GAN model may correspondingly generate the reconstructed PET/MRI image (e.g., rIMG2). Moreover, since the GAN has been trained, it may generate the reconstructed PET/MRI image rIMG2 that is similar to the real PET/MRI image IMG based on the causal relationship(s) learned from the extracted causal feature(s). The use of causal feature(s) extracted from preprocessed input variable(s) to generate synthetic image(s) may help to mitigate the potential loss of important feature(s) in the reconstructed PET/MRI image(s) by providing the GAN model with more information about the underlying causal mechanisms that drive the relationship(s) between the input variable(s) and the outcome variable. This can help the GAN model to generate image(s) that are more faithful to the underlying data and preserve important feature(s), thereby improving the accuracy and robustness of image reconstruction.

In step S206, the image reconstruction device 10 may also use appropriate metrics (e.g., mean square error or peak signal-to-noise ratio (PSNR)) or structural similarity index (SSIM) to evaluate the accuracy and quality of the reconstructed PET/MRI image(s) rIMG1 or rIMG2.

In one embodiment, corresponding to step S206 of the image reconstruction method 20, a pseudocode may comprise:

```
# Step 3: Use GAN for image synthesis
  # Generate synthetic image from input variables (PET sinograms, MRI sequences, patient
  demographics, imaging protocol and scanner characteristics) causal features
  synthetic_image = generate_image
```

In one embodiment, the topology actually enables artificial intelligence (AI) servers on local data network to run medical AI applications for smart medicine, in addition to smart construction. In one embodiment, the image reconstruction device 10 may be installed on an AI server, an imaging device, a computer, or a mobile phone. Alternatively, the image reconstruction device 10 may be implemented using a particular machine and externally connected to an imaging device. The module(s) (e.g., 120, 142, 144, or 160), network(s) (e.g., 660G or 660D), or block(s) (e.g., 544D, 544C1, or 544C2) of the image reconstruction device 10 may be implemented using hardware (e.g., circuit(s)), software or firmware.

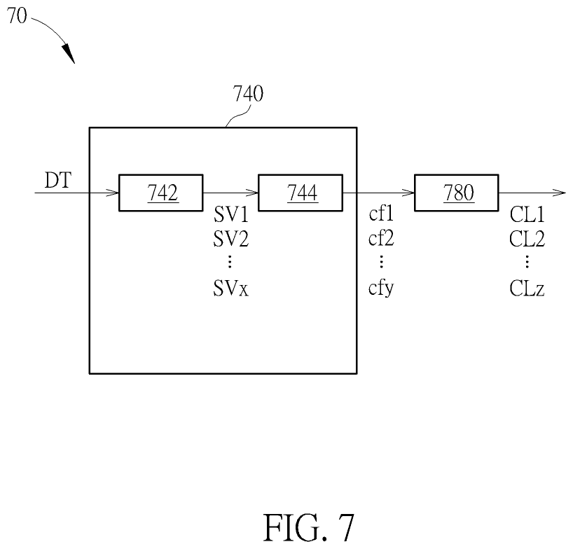

FIG. 7 is a schematic diagram of a priority triage device 70 according to an embodiment of the present invention. The priority triage device 70 may comprise an establishment module 740, a decision analysis module 780 and a judgment module 790. The establishment module 740 may comprise a causal model building module 742 and a CFL module 744.

The causal model building module 742 may be used to receive input data DT and build a causal model, which may comprise state variables SV1 to SVx. The CFL module 744 may be used to extract causal features cf1-cfy from the imaging test. The decision analysis module 780 may be used to receive the causal features cf1-cfy, and output confidence levels CL1 to CLz.

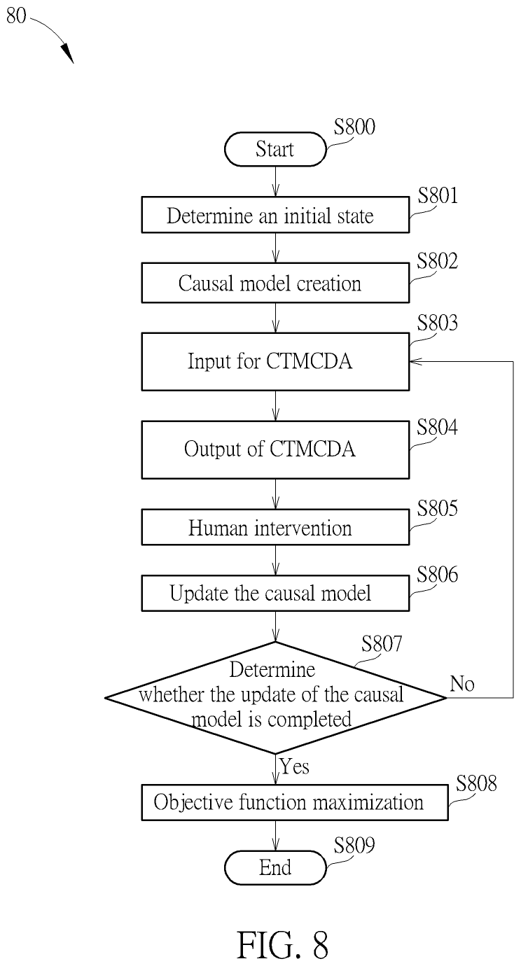

FIG. 8 is a schematic diagram of a priority triage method 80 according to an embodiment of the present invention. The priority triage method 80 is suitable for the priority triage device 70 and may comprise the following steps:

Step S800: Start.

Step S801: Determine an initial state.

Step S802: Causal model creation.

Step S803: Input for continuous time multi-criteria decision analysis (CTMCDA).

Step S804: Output of CTMCDA.

Step S805: Human intervention.

Step S806: Update the causal model.

Step S807: Determine whether the update of the causal model is completed. If complete, proceed to step S808; if not, go to step S803.

Step S808: Maximize the objective function.

Step S809: End.

The priority triage method 80 is further described below. In step S801, a state variable may start from an initial state, which represents medical condition and history of a patient.

In step S802, the causal model building module 742 may use dynamic causal planning graph(s) (DCPG) to create the causal model. In one embodiment, the causal model may comprise CTSCM. In other words, the priority triage method 80 may employ causal AI planning comprising CTSCM.

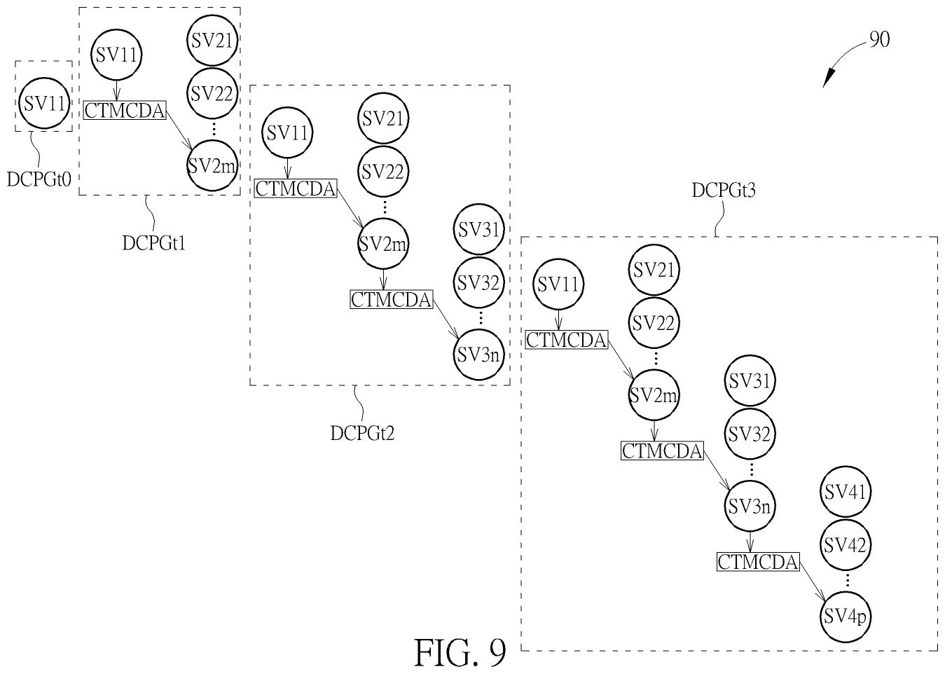

For example, FIG. 9 is a schematic diagram of a CTSCM 90 according to an embodiment of the present invention. In FIG. 9, DCPGs DCPGt0-DCPGt3 at different time instants t0-t3 may comprise state variables SV11, SV21-SV2m, SV31-SV3n, and SV41-SV4p, respectively. A state variable SVx may be implemented using use the state variable(s) SV11, . . . , or SV4p, and the state variable SV11 may be an initial state. FIG. 9 shows that a DCPG (e.g., DCPGt0) only corresponds to one specific moment of the CTSCM 90, while the CTSCM 90 may comprise/correspond to numerous moments. In other words, the CTSCM 90 may be regarded as comprising DCPGs DCPGt0-DCPGt3 to represent the CTSCM 90 as a sequence of DCPGs. Each of a sequence of DCPGs DCPGt0-DCPGt3 may represent the state of the system at a given time instant.

In one embodiment, DCPG (e.g., DCPGt1) may represent the causal relationship(s) between different medical/state variables (e.g., SV11 and SV2m). An edge DG of a DCPG may represent/capture the causal relationship(s) between the state variables, and the actions that can be taken to influence the system. In one embodiment, the state variable may comprise/be, for example, a patient's diagnosis, treatment plan(s), and overall health outcome(s). In one embodiment, the state variable may comprise/be, for example, an imaging test, medical condition, medical history, patient diagnosis, treatment plan(s), or overall health outcome(s).

In one embodiment, the DCPG may be a planning graph that allows the causal graph to evolve during planning time. In other words, in a DCPG, the causal relationship(s) between state variables is/are not fixed, but may change over time based on the action(s) that is/are taken. In one embodiment, DCPG may replace the traditional planning tree and be used in traditional AI planning.

In one embodiment, the selection of an imaging test may be viewed as an action that affects the causal relationship(s) between these state variables. Selecting an appropriate imaging test may influence the causal relationship(s) between different medical/state variables, such as the patient's diagnosis, treatment plan, or overall health outcome(s). This is because the accuracy of the information provided by the imaging test may impact subsequent action(s) taken by healthcare providers.

In step S802, the CFL module 744 may extract causal feature(s) of a precondition state variable from/by the previous imaging test (referred to as a first imaging test). In one embodiment, a state variable (e.g., SVx) may comprise at least one causal feature (e.g., cfy).

In one embodiment, causal feature(s) may refer to any relevant information obtained/extracted from previous imaging test(s). In one embodiment, causal feature(s) extracted from imaging test(s)/result(s) comprise anatomy-based feature(s), contrast-based feature(s), texture-based feature(s), shape-based feature(s), or spatial-based feature(s). In one embodiment, anatomy-based features may refer to features that capture the anatomical structures present in an imaging test, such as the size, density, or location of bones, organs, or blood vessels. In one embodiment, contrast-based features may refer to features that capture the contrast differences in an imaging test, such as the presence of soft tissue or fluid in an image. In one embodiment, texture-based features may refer to features that capture the texture or pattern of an image, such as the presence of microcalcifications or lesions. In one embodiment, shape-based features may refer to features that capture the shape(s) and contour(s) of object(s) in an image, such as the curvature of bones or organs. In one embodiment, spatial-based features may refer to features that capture the spatial relationship(s) between object(s) in an image, such as the relative positions of bones or organs.

In one embodiment, a causal feature may comprise, for example, medical imaging test selection factor(s) or the presence or absence of certain medical condition(s) or abnormality/abnormalities. In one embodiment, a medical imaging test selection factor may comprise, for example, patient's medical history (which, for example, comprises any relevant past medical conditions, surgeries, or procedures), symptom(s) (each of which, for example, comprises the nature, severity, or duration of the patient's symptom), patient's current medical state (e.g., the patient's current physiological or clinical status), allergy/allergies (e.g., any known allergies or adverse reactions to medication(s) or contrast agent(s)), pregnancy (i.e., whether the patient is pregnant or not), the patient's age, imaging test risk(s) and benefit(s) (which, for example, comprise contrast agent exposure, radiation exposure, or invasiveness), cost (e.g., the financial cost of an imaging test or any associated follow-up procedures), or the availability/accessibility of (necessary) imaging equipment and (trained) personnel.

In one embodiment, in step S802, the CFL module 744 may use a CFL algorithm to extract causal feature(s). In one embodiment, the CFL algorithm may be an unsupervised CFL algorithm, a CFL neural network, or a CFL neural network in the form of CTSCM.

For example, the CFL module 544 shown in FIG. 5 may implement the CFL module 744. In one embodiment, the density estimation block 544D of the CFL module 544 may be used to receive the data 5X, 5Y and estimate probability density function(s) of the data 5X, 5Y. The data 5X or 5Y may be, for example, one or more of the state variables SV1-SVx, SV11-SV4p. For example, the data 5X may be/comprise medical condition(s) and the data 5Y may be imaging test(s)/result(s). In one embodiment, the clustering block 544C of the CFL module 544 is used to divide the data 5X or 5Y into different clusters. The clustering of the data 5X or 5Y may be performed according to causal feature(s), so that the CFL module 544 may use the CFL algorithm to extract causal feature(s) (e.g., CF or cf) from the data 5X or 5Y.

In one embodiment, the priority triage method 80 may involve modifying the CFL algorithm used by the CFL module 544 for CTSCM, and may further comprise steps S600-S610.

In one embodiment, the establishment module 740 (e.g., the CFL module 744) may incorporate causal feature(s) into DCPG(s) to help to capture the underlying causal relationship(s) between the medical condition(s) and imaging test(s)/result(s), leading to more accurate and efficient decision-making. In one embodiment, medical conditions may comprise, for example, any health conditions/diseases that may be present/suspected in a patient (e.g., infections, injuries, chronic illnesses, or other medical issues that can affect a patient's health).

In step S803, the CTMCDA of the decision analysis module 780 may takes as input the causal feature(s) extracted by/from the previous imaging test (or medical imaging test selection factor(s)). In other words, the priority triage method 80 may use at least one DCPG and CTMCDA in at least one CTSCM.

In one embodiment, once the causal feature(s) has/have been extracted, a causal inference algorithm may be used to identify the underlying cause-and-effect mapping(s). In one embodiment, a Structure Causal Modeling framework may be used as the causal inference algorithm to estimate the causality between medical condition(s) and imaging test(s)/result(s).

In step S804, the CTMCDA of the decision analysis module 780 may calculate a confidence level (e.g., CLz) for each action alternative or a confidence level (e.g., CL1) for each imaging test (e.g., each second imaging test). In one embodiment, action alternative(s) may be effect state variable(s). In one embodiment, a confidence level may represent the need for the next imaging test (referred to as a second imaging test), whether the next imaging test is necessary, or the degree of necessity of performing the next imaging test. The confidence level may be related to or based on the relevant medical imaging test selection factor(s), or the causal feature(s) of the previous imaging test(s).

For example, if a patient has just undergone a CT scan (serving as a first imaging test) with contrast and a brain tumor was identified, the CTMCDA might calculate a high confidence level for a follow-up MRI (serving as a second imaging test) to further assess the size and location of the tumor. On the other hand, if the previous CT scan shows no abnormalities, the CTMCDA may calculate a lower confidence level for a follow-up imaging test (i.e., a second imaging test), unless the patient's medical state or relevant factor(s) have changed.

Once the CTMCDA has provided confidence level(s) for each action alternative in step S804, human expert(s) may intervene to select the best course of action in step S805 based on their medical judgment, expertise, or confidence level (e.g., CLz). Once the best action alternative is determined by human intervention in step S805, the causality between a precondition state variable and an effect state variable may be fixed, and the DCPG may then be used to simulate the effect(s) of subsequent action(s) based on the chosen course of action.

Accordingly, after the human expert(s) selects the best course of action in step S805, the causal model may be updated in step S806 to reflect the effect(s) of the chosen action. In one embodiment, in step S806, the state variable(s) may be updated according to the selected action; alternatively, a new state variable may be generated. For example, in FIG. 9, at the time instant t1, the DCPG DCPGt1 may comprise the state variables SV11, SV21-SV2m. After the state variable SV2m (serving as a candidate imaging test) is determined/selected from the state variables SV21, . . . , or SV2m or its corresponding action (serving as a second imaging test) in step S805 according to the confidence level(s) of the state variables SV21, . . . , or SV2m, the causal model may be updated. As a result, the DCPG DCPGt2 at the time instant t2 may comprise the state variables SV11, SV21-SV2m, and SV31-SV3n to reflect the state variable(s) (e.g., the newly generated state variable SV31 . . . or SV3n) corresponding to the candidate imaging test (e.g., the state variable SV2m).

In step S807, the priority triage device 70 (e.g., the judgment module 790) may judge/determine whether the update of the causal model is completed. If the update of the causal model is completed, steps S803-S806 are repeated, and the updated causal model is used as a new starting point for the next iteration. For example, the DCPG DCPGt2 at the time instant t2 may comprise the DCPG DCPGt1 at the time instant t1.

In step S808, the priority triage device 70 (e.g., the judgment module 790) may maximize the objective function. By using DCPG(s) and CTMCDA in the context of CTSCM(s), it is possible to reason about the causal effect(s) of their action(s) on the system over time and make optimal decision(s) that maximize desired objective function(s), such as reducing the number of unnecessary imaging tests while ensuring that the patient receives appropriate care.

In one embodiment, at least part of the priority triage method 80 may be compiled into a pseudocode, which may comprise, for example:

```
// Step 1: Initial state
state = initialize_state( )
// Step 2: Causal model creation
causal_model = create_causal_model(state)
// Loop over imaging tests
for each imaging_test in imaging_tests:
  // Step 3: CTMCDA input
  // based on the relevant medical imaging test selection factors and the causal features of the
  previous imaging test
  selection_factors = relevant_selection_factors( )
  causal_features = extract_causal_features(imaging_test)
  // Step 4: CTMCDA output
  confidence_levels = calculate_confidence_levels(causal_features, causal_model)
  // Step 5: Human intervention
  selected_action = human_intervention(confidence_levels)
  // Step 6: Update causal model
  state = update_state(selected_action)
  causal_model = update_causal_model(selected_action, causal_model)
// Step 8: Objective function maximization
maximize_objective_function(causal_model)
```

In one embodiment, i, j, k, m, n, p, x, y, or z may be positive integers, but are not limited thereto.

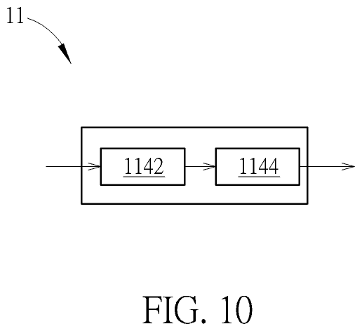

FIG. 10 is a schematic diagram of a causal device 11 according to an embodiment of the present invention. The causal device 11 may be implemented using the image reconstruction device 10 shown in FIG. 1 or the priority triage device 70 shown in FIG. 7.

The causal device 11 may comprise a causal module 1142 and a CFL module 1144. The causal module 1142 may be used to identify/exploit/utilize causal relationship(s) between multiple variables. The causal module 1142 may be implemented using the causal inference module 142 shown in FIG. 1 or the causal model building module 742 shown in FIG. 7.

The CFL module 1144 may be used to extract at least one causal feature of/from one of the multiple variables. The CFL module 1144 may be implemented using the CFL module 144 shown in FIG. 1 or the CFL module 744 shown in FIG. 7. The CFL module 1144 may use a CFL algorithm to extract causal feature(s). For example, the CFL module 1144 may be implemented using the CFL module 544 shown in FIG. 5.

In summary, PET data (e.g., PET sinogram(s) or PET image(s)) and MRI data (e.g., MRI sequence(s) or MRI image(s)) may be considered as input variables in CTSCM that describe the causal relationships between PET data, MRI data, and the reconstructed PET/MRI image. The CTSCM may be used to model the dynamics of the system and capture the causal relationships between these variables, allowing for more accurate and robust image reconstruction.

In summary, the use of DCPG(s) and CTMCDA model in the context of CTSCM(s) is a valid and doable approach for reasoning about the causal effects of medical imaging tests and making optimal decisions. By representing the CTSCM as a sequence of DCPGs, it becomes possible to model the changing causal relationship(s) between medical status variable(s) over time and evaluate the potential impact of different imaging tests on the causal relationship(s). The CTMCDA algorithm may then be used to calculate a confidence level for each imaging test, based on the relevant medical imaging test selection factors or the causal features of the previous imaging test. Incorporating causal features into the DCPGs can help to capture the underlying causal relationship(s) between the medical conditions and imaging test results, leading to more accurate and efficient decision-making. Therefore, the present invention may help healthcare providers make informed decisions about which imaging tests to order for their patients, based on the best available evidence and clinical judgment.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A causal device, comprising:
a causal module, configured to identify or utilize causal relationships between a plurality of variables; and
a causal feature learning module, coupled to the causal module, configured to extract at least one first causal feature of one of the plurality of variables.

2. The causal device of claim 1, wherein the causal device is an image reconstruction device,
the causal module identifies causal relationships between a plurality of input variables of the plurality of variables and a reconstructed image of the plurality of variables,
the causal feature learning module extracts at least one causal feature from each of the plurality of input variables, and
the causal device further comprises a reconstruction module configured to generate the reconstructed image based on the plurality of causal features.

3. The causal device of claim 1, wherein one of a plurality of input variables of the plurality of variables corresponds to a Positron Emission Tomography (PET) sinogram, a Magnetic Resonance Imaging (MRI) sequence, patient demographics, an imaging protocol, or a scanner characteristic.

4. The causal device of claim 1, wherein the causal device further comprises:
a preprocessing module, configured to preprocess or convert at least one first input variable data into at least one second input variable data, wherein the at least one second input variable data comprises a plurality of input variables of the plurality of variables, and preprocessing performed by the preprocessing module comprises attenuation correction, motion correction, registration, standardization, or normalization.

5. The causal device of claim 1, wherein the causal module applies a continuous time structured equation modeling framework to identify causal relationships between a plurality of input variables of the plurality of variables and a reconstructed image of the plurality of variables, and
a plurality of causal features of the plurality of input variables are combined and inputted to a reconstruction module of the causal device to generate a reconstructed image based on the plurality of causal features using a Generative Adversarial Network (GAN).

6. The causal device of claim 1, wherein the causal feature learning module comprises:
a density estimation block, configured to estimate a plurality of probability density functions of the plurality of variables; and
a clustering block, coupled to the density estimation block, configured to divide the plurality of variables into different clusters according to the plurality of probability density functions so as to extract the at least one first causal feature.

7. The causal device of claim 1, wherein the causal feature learning module extracts the at least one first causal feature based on a causal feature learning algorithm, the causal feature learning algorithm comprises at least one parameter changing over time, an error model changing over time, a time-dependent loss function, or a time-dependent regularization term.

8. The causal device of claim 1, wherein the causal device is a priority triage device, each of the plurality of variables is a first state variable,
the causal module utilizes the causal relationships between the plurality of first state variables to create a first causal model,
the causal feature learning module extracts the at least one first causal feature from a first imaging test of the first causal model, the first imaging test is an imaging test that has been finished,
the causal device further comprises a decision analysis module configured to generate at least one confidence level corresponding to at least one second imaging test based on the at least one causal feature, and each of the at least one second imaging test is an imaging test that has not yet been performed.

9. The causal device of claim 1, wherein each of the plurality of variables is a first state variable, and one of the plurality of first state variables comprises an imaging test, medical condition, medical history, patient diagnosis, a treatment plan, or an overall health outcome.

10. The causal device of claim 1, wherein after a candidate imaging test is selected from at least one second imaging test, a first causal model is updated to become a second causal model, the second causal model reflects at least one second state variable corresponding to the candidate imaging test.

11. A causal method, for a causal device, comprising:
identifying or utilizing causal relationships between a plurality of variables; and
extracting at least one first causal feature of one of the plurality of variables.

12. The causal method of claim 11, wherein identifying the causal relationships between the plurality of variables comprises identifying causal relationships between a plurality of input variables of the plurality of variables and a reconstructed image of the plurality of variables, and
after at least one causal feature is extracted from each of the plurality of input variables, the reconstructed image is generated based on the plurality of causal features.

13. The causal method of claim 11, wherein one of a plurality of input variables of the plurality of variables corresponds to a Positron Emission Tomography (PET) sinogram, a Magnetic Resonance Imaging (MRI) sequence, patient demographics, an imaging protocol, or a scanner characteristic.

14. The causal method of claim 11, further comprising:
preprocessing or converting at least one first input variable data into at least one second input variable data, wherein the at least one second input variable data comprises a plurality of input variables of the plurality of variables, and preprocessing being performed comprises attenuation correction, motion correction, registration, standardization, or normalization.

15. The causal method of claim 11, wherein identifying the causal relationships between the plurality of variables comprises the causal module applies a continuous time structured equation modeling framework to identify causal relationships between a plurality of input variables of the plurality of variables and a reconstructed image of the plurality of variables, and
after a plurality of causal features of the plurality of input variables are combined, a reconstructed image is generated based on the plurality of causal features using a Generative Adversarial Network (GAN).

16. The causal method of claim 11, further comprising:
estimating a plurality of probability density functions of the plurality of variables; and
dividing the plurality of variables into different clusters according to the plurality of probability density functions so as to extract the at least one first causal feature.

17. The causal method of claim 11, wherein the at least one first causal feature is extracted based on a causal feature learning algorithm, the causal feature learning algorithm comprises at least one parameter changing over time, an error model changing over time, a time-dependent loss function, or a time-dependent regularization term.

18. The causal method of claim 11, wherein each of the plurality of variables is a first state variable,
utilizing the causal relationships between the plurality of variables comprises utilizing the causal relationships between the plurality of first state variables to create a first causal model,
after the at least one first causal feature is extracted from a first imaging test of the first causal model, at least one confidence level corresponding to at least one second imaging test is generated based on the at least one causal feature, the first imaging test is an imaging test that has been finished, and each of the at least one second imaging test is an imaging test that has not yet been performed.

19. The causal method of claim 11, wherein each of the plurality of variables is a first state variable, and one of the plurality of first state variables comprises an imaging test, medical condition, medical history, patient diagnosis, a treatment plan, or an overall health outcome.

20. The causal method of claim 11, wherein after a candidate imaging test is selected from at least one second imaging test, a first causal model is updated to become a second causal model, the second causal model reflects at least one second state variable corresponding to the candidate imaging test.
